(19) United States
(12) Patent Application Publication
(10) Pub. No.: US 2012/0290086 A1
(43) Pub. Date: Nov. 15, 2012

(54) INTRAOCULAR CAPSULAR TENSION RINGS

(75) Inventors: Boris E. Malyugin, Moscow (RU); Lawrence Laks, Bellevue, WA (US); Kenneth J. Wiljanen, Redmond, WA (US)

(73) Assignee: MICROSURGICAL TECHNOLOGY, INC., Redmond, WA (US)

(21) Appl. No.: 13/288,789

(22) Filed: Nov. 3, 2011

Related U.S. Application Data
(60) Provisional application No. 61/409,848, filed on Nov. 3, 2010.
(60) Provisional application No. 61/415,760, filed on Nov. 19, 2010.

(51) Int. Cl. A61F 2/16

(52) U.S. Cl. 623/6.39

(57) ABSTRACT

Arcuate arms each approximately semi-circular extend generally oppositely from a central fixation element. Together the arms and integral fixation element form a capsular tension ring to apply outward pressure along the equator of an ocular lens capsule during surgery. The ring can be loaded into an insertion tube by pulling on the central fixation element such that the arms are drawn into the tube together. Similarly, the arms are expelled together from the tube during insertion into the capsule so as to equalize the force applied to the capsule during insertion of the ring.

### CROSS-REFERENCE(S) TO RELATED APPLICATIONS

This application claims the benefit of U.S. Provisional Application 61/409,848, filed Nov. 3, 2010, and U.S. Provisional Application 61/415,760, filed Nov. 19, 2010, the disclosures of which are expressly incorporated by reference herein.

### BACKGROUND

Capsular tension rings (CTRs) of the general type with which the present invention is concerned are shown in Nishi U.S. Pat. No. 6,319,282, and Cionni U.S. Pat. No. 5,843,184, both of which are expressly incorporated by reference herein.

In general, known capsular tension rings are round spring clips of an exaggerated “C” shape encompassing almost 360°. The rings are formed of resilient biocompatible materials and are sized to fit within an ocular lens capsule, near the equator, in a compressed state to apply outward-directed pressure. A CTR can be very useful in stabilizing the lens capsule (commonly referred to as the “bag”) during eye surgery, such as cataract removal, particularly for patients with missing or weak zonules that normally would stably connect the capsule to the sclera and center the bag. Often, the adjacent ends of the CTR are curved or bent inward and are formed with eyelets that are useful for manipulating the CTR into the desired centered position. It is known to insert a CTR by first drawing it endwise into a small diameter injector tube. For example, one of the end eyelets can be hooked or grasped by an inner plunger or piston element effective to pull the CTR endwise into the tube. The tube can be inserted through a small slit in the cornea and part-way through the anterior opening of the bag formed by capsularhexis. Then, the CTR is ejected lengthwise from the tube and, ideally, will relax to its essentially circular shape at the equator of the bag.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect of the present invention an intraocular capsular tension ring has a central fixation element adapted to be received by an insertion device. Resilient arcuate arms extend generally oppositely from the fixation element, such arms forming an arc to engage along the equatorial region of an ocular lens capsule. The fixation element and arms are constructed and arranged relatively to be loaded into the insertion device by pulling on the central fixation element and thereby draw the arms into the insertion device together. Similarly, when desired the arms are expelled together from the insertion device into the capsule so as to equalize the force applied to the capsule as the arms return to the arcuate condition inside the capsule.

Additional features of the invention adapt a ring for anchoring, such as by suture, to the structure surrounding the lens capsule, and/or to reinforce or stabilize areas of the capsule.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 (prior art) is an enlarged, diagrammatic top plan of a capsular tension ring (“CTR”) of the general type with which the present invention is concerned, and

FIG. 2 (prior art) is an enlarged, fragmentary, diagrammatic top plan of the CTR of FIG. 1 during insertion into the lens capsule of an eye during surgery;

FIG. 3 is an enlarged, diagrammatic top plan of a CTR in accordance with a first embodiment of the present invention;

FIGS. 4A-4E are enlarged, fragmentary, diagrammatic top plans of the CTR of FIG. 3 illustrating different positions as such CTR is loaded into an insertion tube;

FIGS. 5A-5C are enlarged, fragmentary, diagrammatic top plans of the CTR of FIG. 3 illustrating different positions as such CTR is ejected from an insertion tube during surgery;

FIG. 6 is an enlarged, diagrammatic top plan of a CTR in accordance with a second embodiment of the present invention;

FIG. 7A is a further enlarged, fragmentary, diagrammatic top plan of a CTR in accordance with a third embodiment of the present invention, and FIG. 7B is a diagrammatic top plan of such third embodiment on a smaller scale;

FIG. 8 is an enlarged, fragmentary, diagrammatic top plan of a CTR in accordance with a fourth embodiment of the present invention;

FIG. 9A is an enlarged, fragmentary, diagrammatic top plan of a CTR in accordance with a fifth embodiment of the present invention, and FIGS. 9B-9D are enlarged, fragmentary, diagrammatic top plans of the CTR of FIG. 9 illustrating different positions as such CTR is loaded into an insertion tube and then ejected and fixed in place during surgery;

FIG. 10 is an enlarged, fragmentary, diagrammatic top plan of a capsular tension “hook” in accordance with the present invention; and FIG. 10A is an enlarged, fragmentary, diagrammatic top plan of a modified form of such hook;

FIG. 11 is an enlarged, fragmentary, diagrammatic top plan of a capsular tension hook in accordance with the present invention, after insertion into a lens capsule;

FIGS. 11A-11C are enlarged, fragmentary, diagrammatic side elevations of the hook of FIG. 11, illustrating alternative shapes for aspects of such a hook in profile;

FIG. 12 is a diagrammatic top plan of a capsular tension hook in accordance with the present invention when fixed in place during surgery;

FIG. 13 is an enlarged, fragmentary, diagrammatic top plan of a CTR in accordance with a another embodiment of the present invention, and FIGS. 13A-13D are enlarged, fragmentary, diagrammatic top plans of the CTR of FIG. 13 illustrating different positions as such CTR is loaded into an insertion tube;

FIG. 14 is an enlarged, diagrammatic top plan of the CTR of FIG. 13 when fixed in place during surgery;

FIG. 15 is an enlarged, diagrammatic top plan of a CTR in accordance with yet another embodiment of the present invention, and FIG. 15A is a further enlarged, diagrammatic top plan of a CTR in accordance with FIG. 15;

FIG. 16 is an enlarged, top perspective of a CTR in accordance with a still another embodiment of the present invention,

FIG. 17 is a top plan thereof,

FIG. 18 is front elevation thereof,

FIG. 19 is a side elevation thereof, and

FIG. 20 is a fragmentary sectional view thereof taken along line 20-20 of FIG. 17.

### DETAILED DESCRIPTION

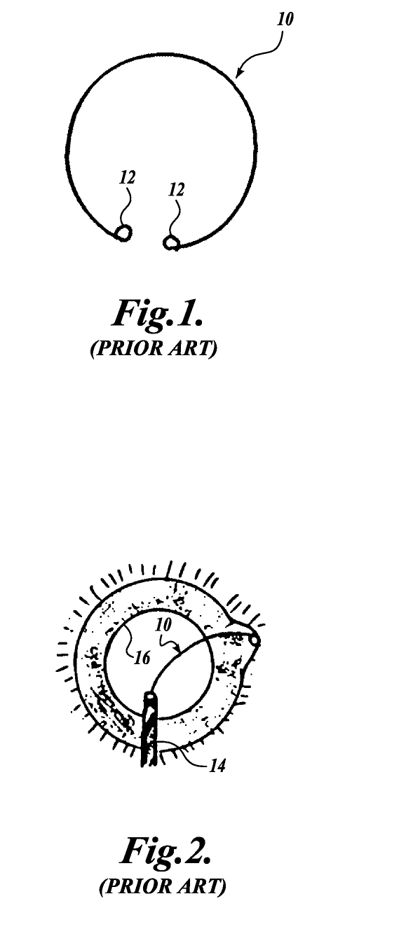

As seen in FIG. 1, a representative conventional capsular tension ring (CTR) 10 has the usual, essentially circular shape with an eyelet 12 at each end. With reference to FIG. 2, a CTR in accordance with FIG. 1 can be preloaded endwise into an injector tube 14. Tube 14 is inserted through a small corneal slit to a location close to or partially through the anterior capsularhexis edge 16, whereupon the CTR is projected endwise from the tube 14. The distal end of the CTR will engage against the interior of the capsular bag and necessarily apply localized force on the inner portion of the capsule. In an extreme example, this can lead to damage of the zonular fibers, which may already be weakened or providing uneven forces on the bag, or even rupture of the bag itself.

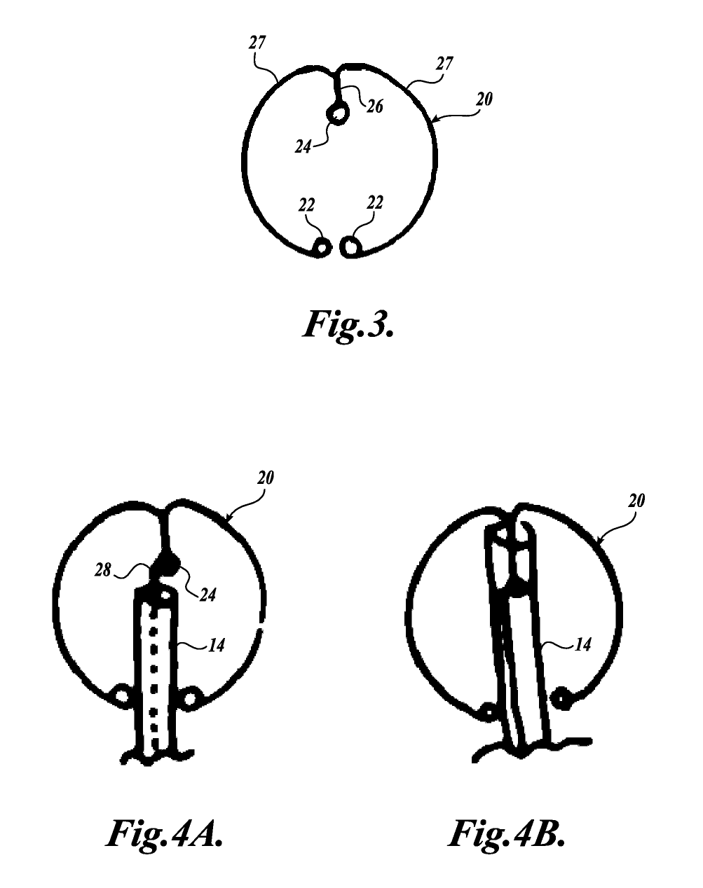

With reference to FIG. 3, in accordance with one embodiment of the present invention, a modified CTR 20 has the same aggregate arcuate extent as known CTRs, and can have similar inwardly formed end eyelets 22. In the modified CTR 20, however, an additional eyelet or fixation element 24 is provided in the central region of the arc of the CTR, such as directly across from the opening between the eyelets 22. In one embodiment the fixation element 24 can be spaced inward by a short stem section 26, with oppositely projecting arms 27 forming the substantially circular periphery of the CTR. All parts can be in the same plane.

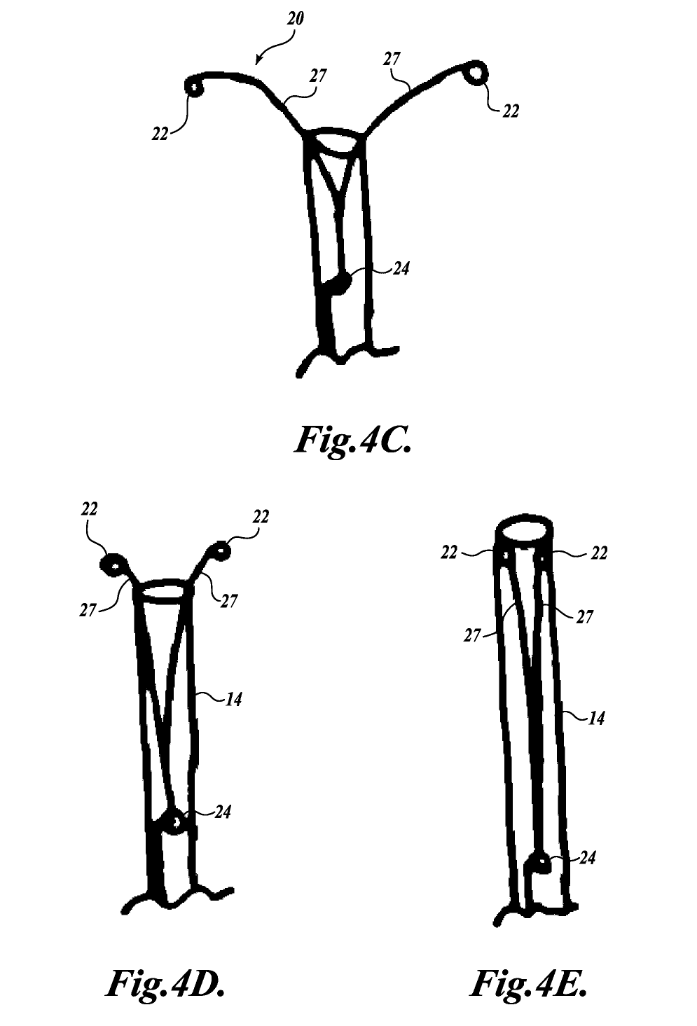

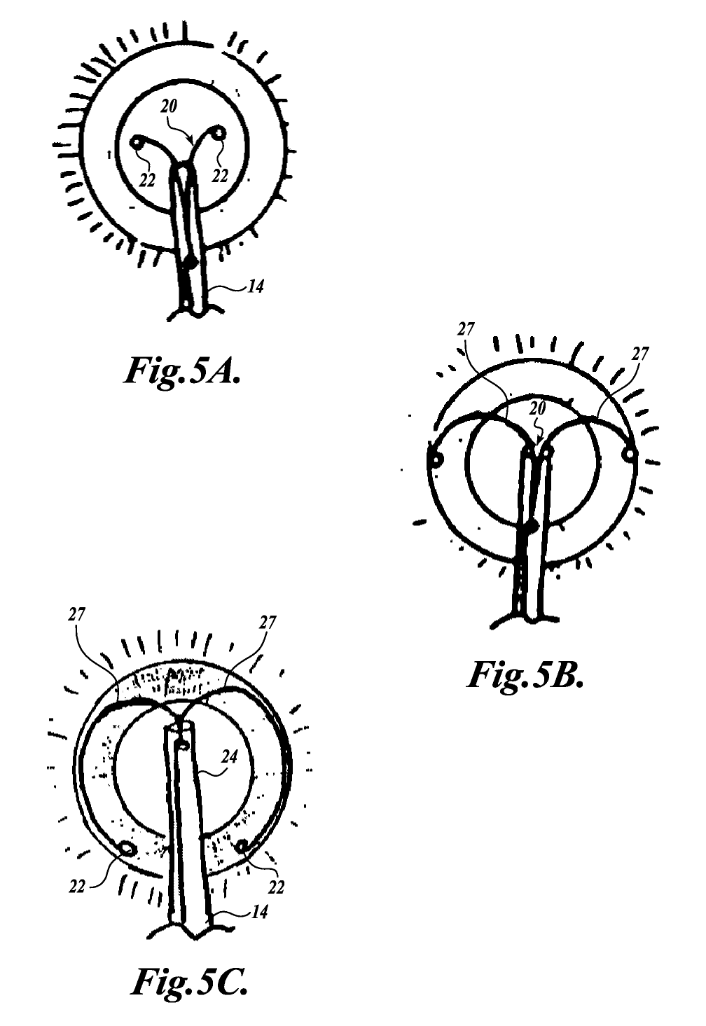

As represented in FIG. 4A, rather than loading the CTR 20 endwise into an injector tube 14, a piston or plunger hook 28 of an insertion device is fitted through the central eyelet 24, then, as represented in FIG. 4B, the CTR 20 is drawn into the tube from the central portion. As represented in FIGS. 4C-4E, as the CTR 20 is further withdrawn, the oppositely extending arms 27 between the end eyelets 22 and the center eyelet or fixation element 24 are simultaneously pulled into the tube until they lie fully therein (FIG. 4E). With reference to FIG. 5A, the injector tube 14 is positioned in the capsular bag conventionally prior to expelling the CTR into the bag. As seen in FIG. 5B and FIG. 5C, the curved ends of the CTR arms 27 are shaped to slide along the inner periphery of the capsular bag so that force applied to the capsular wall is equalized and the bag has less tendency to shift. As full insertion is approached, more and more of the inner periphery of the bag is gently engaged and urged outward by the oppositely extending arms 27 of the CTR 20, until the center eyelet 24 or other fixation device can be disengaged and the capsule will be stabilized with the CTR in place in the equatorial region of the bag.

An alternative procedure is to draw the central eyelet or fixation point into the injector tube 14 without drawing in the oppositely extending arms of the CTR. For example, with the parts in approximately the position shown in FIG. 4B, the CTR and tube can be passed through the slit in the cornea. The arms will collapse toward each other as they pass through the slit. Thereafter, the CTR can be manipulated to the deployed condition in the equatorial region of the bag.

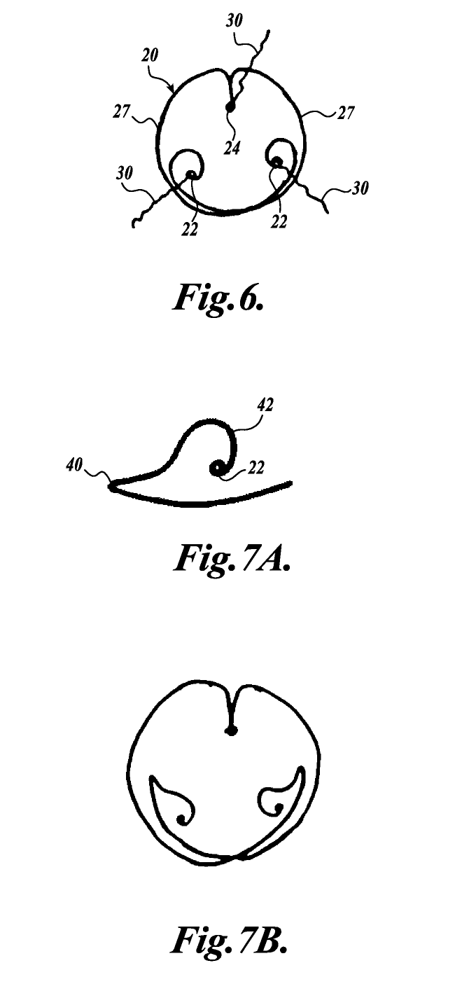

In the modification of FIG. 6, the CTR 20 has longer arms 27 projecting oppositely from the central eyelet or fixation element 24. Such arms can be long enough so as to overlap sufficiently that suture 30 can be used to secure the CTR and capsular bag held thereby to the scleral wall at approximately equiangular positions, i.e., approximately 120° apart. Also, the end portions of the CTR near the end eyelets 22 can be more tightly curved to assure that a blunt curved section of the CTR is presented to the inner surface of the bag during implanting. In this embodiment, the overlapping end portions can be angled or curved outward (toward the viewer in FIG. 6) and hook over the capsularhexis edge or at least be positioned at the anterior side so that the eyelets 22 can be sutured to the sclera without the suture penetrating the bag.

FIGS. 7A and 7B illustrate another embodiment where the end portions of the CTR are designed for easier, smoother sliding along the inner periphery of the capsular bag. Each end can have a reflex bend 40 turned inward with a rounded end portion 42 leading to an end eyelet 22. The corresponding CTR has a shape such that the ends turn quickly away from each other and move in an arc as they are expelled from the injector tube, ultimately to approximately the shape shown in FIG. 7B.

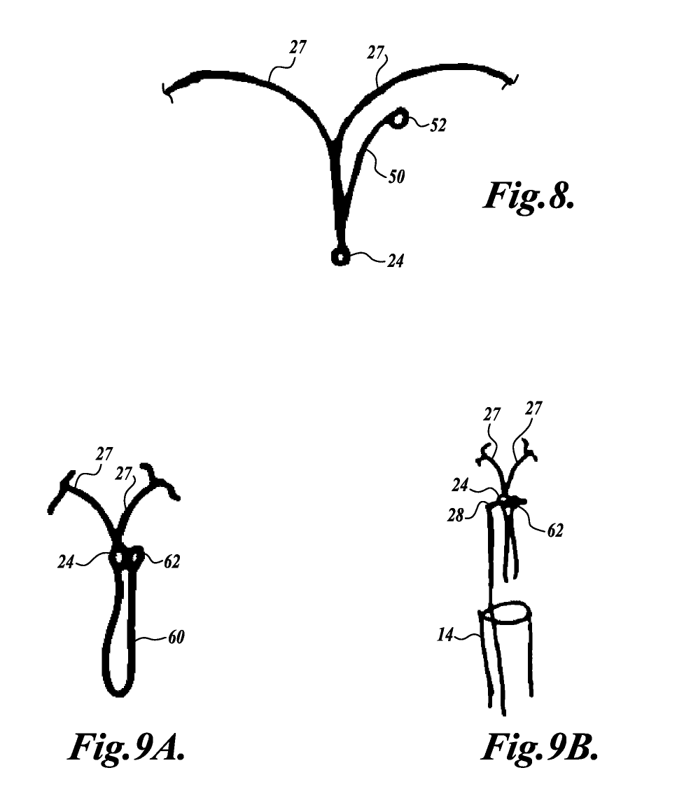

In the embodiment of FIG. 8, an additional stem 50 of the CTR material leads from the fixation point 24 to an offset eyelet 52 that can be used for suturing to the scleral wall. Stem 50 can be angled or curved outward to position eyelet 52 at the anterior of the capsularhexis edge for suturing directly to the sclera.

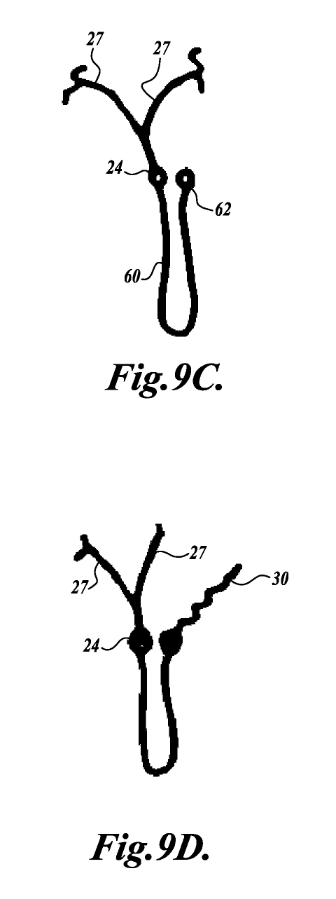

In the embodiment of FIGS. 9A-9D, a loop 60 of the CTR material extends from the fixation element 24 and has an additional eyelet 62. As represented in FIG. 9B, both eyelets can be grabbed by the injector hook 28 for loading inside an injector tube 14. When projected to the condition of FIG. 9C, the separate eyelet 62 can be placed on top of the anterior capsule such that the loop 60 will extend from inside the capsule to the anterior side and not lie in the equatorial plane. As represented in FIG. 9D, the separate eyelet 62 then can be used for fixation from the anterior side of the bag by a length of suture 30, like the previously described embodiments that are intended for scleral suturing.

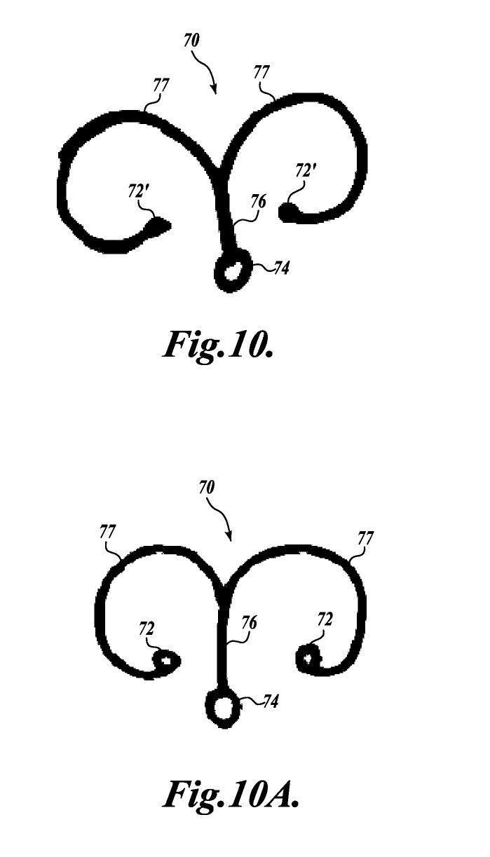

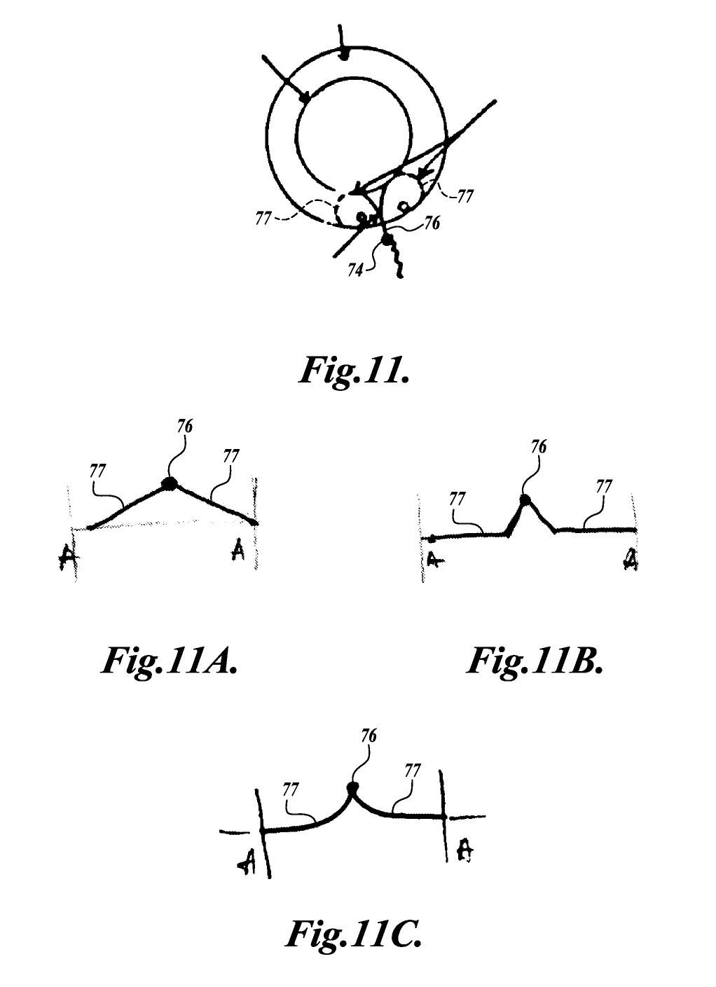

FIGS. 10-12 illustrate a capsular tension “hook” 70 which is designed to engage and stabilize only a portion of a capsular bag, such as in the area of a zonular defect (weak zonules, ruptured zonules, missing zonules). The hook 70 has an appearance somewhat similar to the CTR previously described, namely, a central eyelet 74 or other fixation element and oppositely curved arms 77 leading to blunt ends 72′ (FIG. 10) or eyelets 72 (FIG. 10A). However, the aggregate shape of the hook in the relaxed condition is not essentially circular, and is only a small part of the equatorial circumference of a lens capsular bag. The hook 70 can be withdrawn into an injector tube by means of the central fixation element 74. As shown in FIG. 11, the hook can be projected and manipulated so that the oppositely curved arms extend into the capsular bag and engage a segment at or near the equatorial region, with the central stem 76 and eyelet 74 positioned at the anterior side of the bag where suture stabilization can be used.

More specifically, FIG. 11 is a diagrammatic top plan of the hook 70 after implantation, and FIGS. 11A-11C are diagrammatic side elevations of alternative embodiments. FIG. 11A shows a configuration in which the curved arms 77 are angled upward, out of the central plane, to the stem 76 (and the associated eyelet) which extends generally parallel to the central plane at the anterior side of the bag. The angling is gradual and constant when viewed from the side. In the embodiment of FIG. 11B, the major portion of each arm 77 lies within the central plane so as to engage the interior of the bag at approximately the central plane over a greater surface. Only the portions immediately adjacent to the stem are angled outward, which would be in the area close to the capsularhexis edge. The stem section 76 can extend generally parallel to the central plane, but offset in the anterior direction sufficiently so as to lie outside of the bag, leading to the eyelet which allows the stabilizing suturing to the sclera. The embodiment of FIG. 11C is similar to the embodiment of FIG. 11B, except that the short sections of the arms 77 that diverge out of the central plane are curved when viewed from the side rather than being straight and angled outward. Still, in the embodiment of FIG. 11C, the stem 76 is positioned outside of the bag so that its eye can be sutured to the sclera without penetrating the bag.

Another modification is to form the raised portions 77 and stem 76 at an angle to the central plane (facing outward toward the adjacent capsularhexis edge), rather than having the stem be parallel to the central plane. This construction may adapt the hook for lens capsules having different heights or thicknesses, and the capsularhexis edge can be wedged gently toward the equator of the bag (which may assist in providing a secure fixation and deter CTR rotation). This modification may be used for any embodiment having a part positioned for fixation to the sclera.

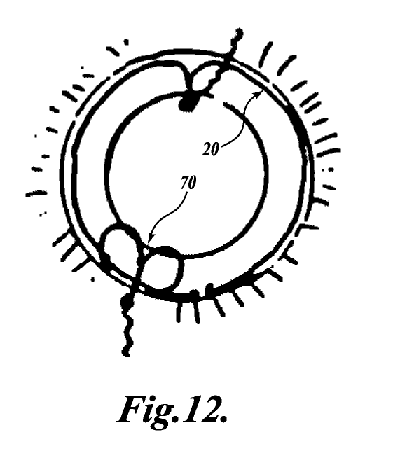

In the implementation of FIG. 12, the capsular tension hook 70 is used in combination with the capsular tension ring 20. The ring applies the desired stabilizing pressure along essentially the entire inner periphery of the bag at approximately the equator, whereas the hook can be used for more localized stabilization in an area of zonular defect.

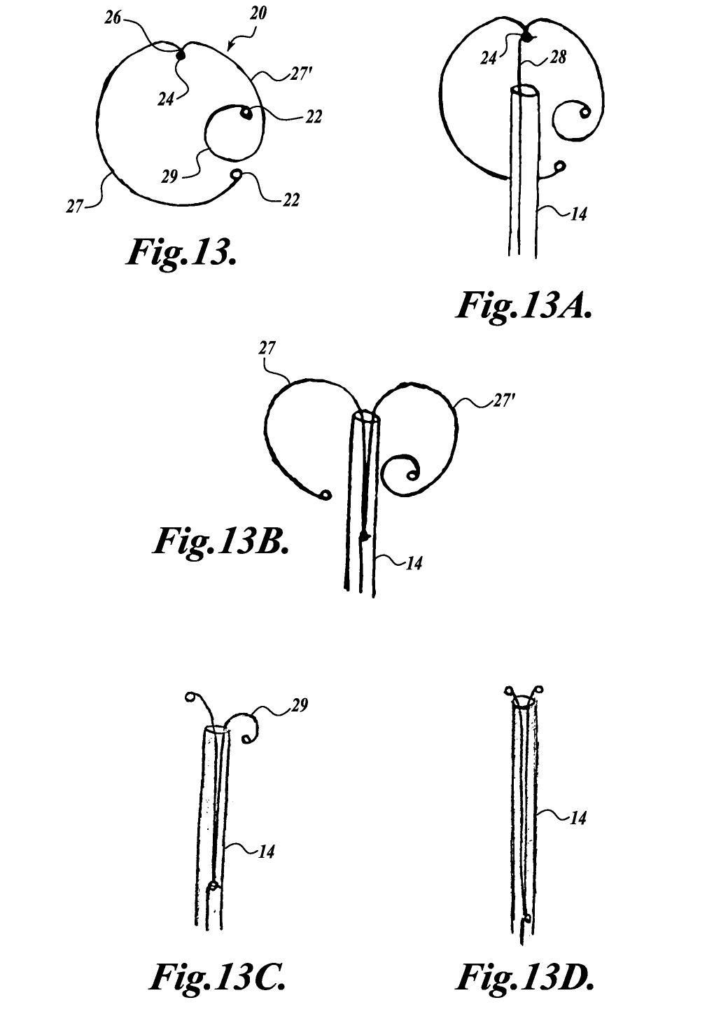

In the embodiment of FIG. 13, one of the curved arm sections 27 of the modified CTR 20 is formed similarly to the embodiment of FIG. 3, except with a greater angular extent. A central fixation element 24 with a short stem 26 is provided along the central portion of the arc of the CTR. The opposite arm 27′ has a larger inwardly curved end portion 29 which approaches 360° in angular extent and leads to an end eyelet 22. The overall length of arm 27′ including the larger curved end portion 29 is about the same as the length of the arm 27. FIGS. 13A-13D illustrate diagrammatically the sequence of drawing a CTR in accordance with FIG. 13 into an insertion tube 14. In FIG. 13A, the central fixation point 24 has been hooked by the reciprocating piston or plunger 28. In FIG. 13B, the plunger has been withdrawn partway, pulling the arms 27, 27′ partway into the tube. In FIG. 13C, the CTR has been drawn farther into the tube, to a point where the end portion 29 is reached and is beginning to uncoil by being forced into the bore of the tube. In FIG. 13D, the arms are almost fully received within the tube, and it can be seen that their overall lengths are approximately equal.

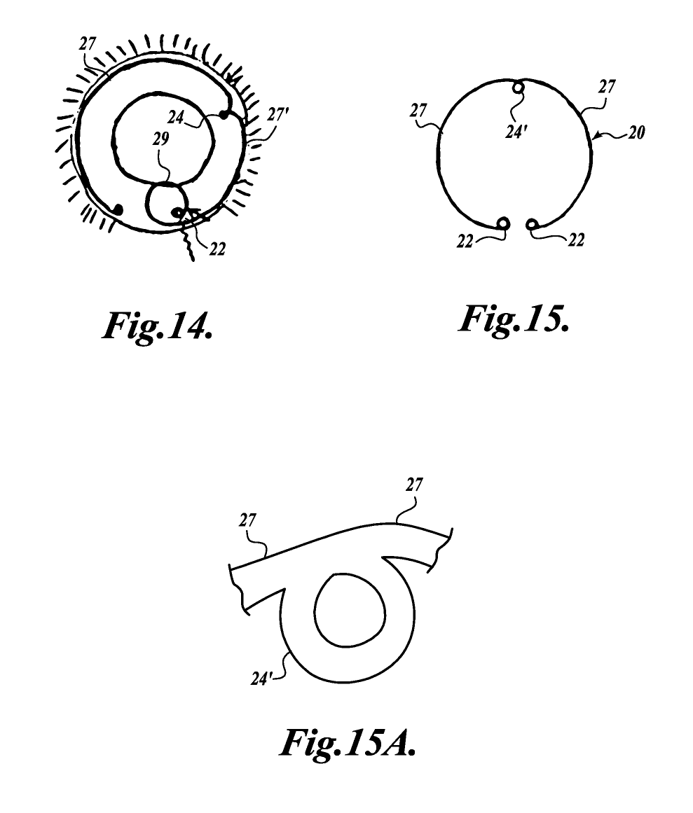

Ejecting the embodiment of FIG. 13 is achieved as for the previously described embodiments. FIG. 14 illustrates the CTR in accordance with FIG. 13 deployed primarily within the capsular bag. Arm 27 engages the inner periphery of the bag at approximately the equator, all the way around to the central fixation point 24. Arm 27′ continues the equatorial engagement, but at least the end portion of the larger curved end 29 is curved or angled out of the central plane such that its eyelet 22 is positioned outside the bag, at the anterior side, for suturing to the sclera.

The geometry of the additional embodiment of FIG. 15 and FIG. 15A is very similar to the geometry of the embodiment of FIG. 3, except that the central fixation point 24′ is formed differently. The CTR 20 still forms a continuous arc of almost 360°, from one end eyelet 22 to the other. Rather than using a stem section between the oppositely extending arms, the central eyelet or fixation point is formed as a small 360° circular portion of the CTR material, as best seen in FIG. 15A. The embodiment of FIG. 15 is deployed within the bag to the same configuration as the embodiment of FIG. 3. Other embodiments may use the fixation point without a stem from the periphery of the CTR.

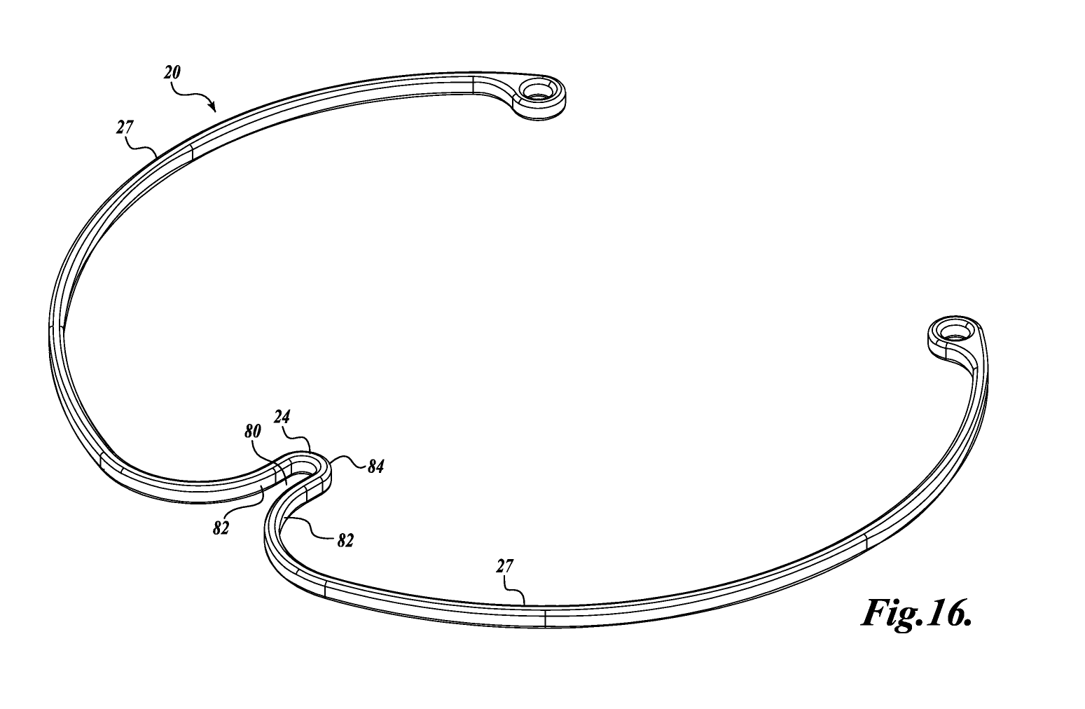

In the embodiment of FIGS. 16-20, the CTR 20 has the central fixation area which forms an outward opening groove 80. The oppositely extending arms 27 have central end portions 82 that are curved inward, generally toward the geometric center of the CTR. Such end portions meet at an inner return bend 84 by means of which the CTR can be hooked by and loaded into an insertion device.

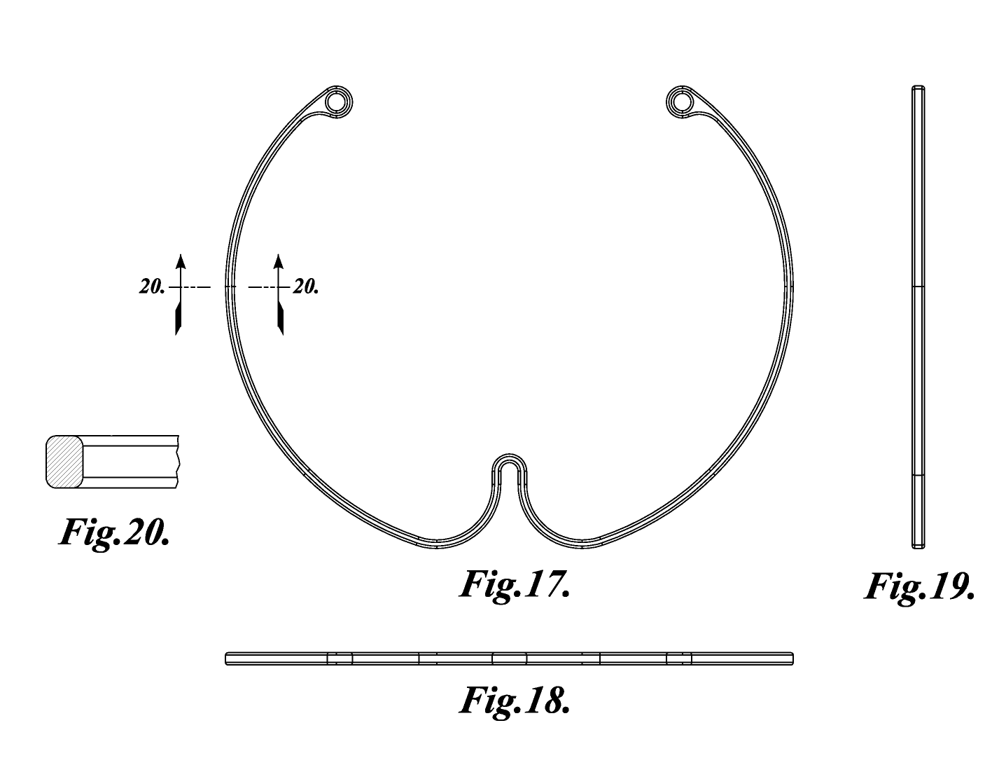

The CTR in accordance with the present invention can be machined from a disc of appropriate resilient medical grade material. Additional details of a representative construction are shown in FIGS. 17-20, such as the cross-sectional shape and approximate relative dimensions.

While illustrative embodiments have been illustrated and described, it will be appreciated that various changes can be made therein without departing from the spirit and scope of the invention.

The embodiments of the invention in which an exclusive property or privilege is claimed are defined as follows:

1. A capsular tension ring for insertion into an ocular lens capsule to apply outward pressure in the area of the equatorial region, said ring having:
a central fixation element adapted to be received by an insertion device; and
two arcuate arms extending generally oppositely from the fixation element, said arms forming an arc to engage along the equatorial region of the capsule, said fixation element and arms being constructed and arranged relatively to be loaded into the insertion device by pulling on the central fixation element and thereby draw the arms into the insertion device together, followed by discharge of the arcuate arms together from the device into the capsule.

2. The ring defined in claim 1, in which the fixation element and the arms are coplanar.

3. The ring defined in claim 1, including a stem section between the fixation element and the arms.

4. The ring defined in claim 3, in which the arms are coplanar and the fixation element is offset out of the plane of the arms when deployed in a capsule.

5. The ring defined in claim 1, in which the fixation element is an eyelet.

6. The ring defined in claim 1, in which the fixation element is a groove formed between adjacent ends of the arms.
